SEQUENCE LISTING (5 sequences)

SEQ ID NO: 1 - protein, 409 aa, Mycoplasma hominis
MSVFDSKFNGIHVYSEIGELETVLVHEPGREIDYITPARLDELLFSAILESHDARKEHQSFVKIMKDRGINVVELTDLVAETYDLASKAAKEEFIETFLEETVPVLTEANKKAVRAFLLSKPTHEMVEFMMSGITKYELGVESENELIVDPMPNLYFTRDPFASVGNGVTIHFMRYIVRRRETLFARFVFRNHPKLVKTPWYYDPAMKMPIEGGDVFIYNNETLVVGVSERTDLDTITLLAKNIKANKEVEFKRIVAINVPKWTNLMHLDTWLTMLDKNKFLYSPIANDVFKFWDYDLVNGGAEPQPQLNGLPLDKLLASIINKEPVLIPIGGAGATEMEIARETNFDGTNYLAIKPGLVIGYDRNEKTNAALKAAGITVLPFHGNQLSLGMGNARCMSMPLSRKDVKW

SEQ ID NO: 2 - protein, 408 aa, Mycoplasma hominis
SVFDSKFNGIHVYSEIGELETVLVHEPGREIDYITPARLDELLFSAILESHDARKEHQSFVKIMKDRGINVVELTDLVAETYDLASKAAKEEFIETFLEETVPVLTEANKKAVRAFLLSKPTHEMVEFMMSGITKYELGVESENELIVDPMPNLYFTRDPFASVGNGVTIHFMRYIVRRRETLFARFVFRNHPKLVKTPWYYDPAMKMPIEGGDVFIYNNETLVVGVSERTDLDTITLLAKNIKANKEVEFKRIVAINVPKWTNLMHLDTWLTMLDKNKFLYSPIANDVFKFWDYDLVNGGAEPQPQLNGLPLDKLLASIINKEPVLIPIGGAGATEMEIARETNFDGTNYLAIKPGLVIGYDRNEKTNAALKAAGITVLPFHGNQLSLGMGNARCMSMPLSRKDVKW

SEQ ID NO: 3 - protein, 417 aa, Mycoplasma hominis
MGGSHHHHHSVFDSKFNGIHVYSEIGELETVLVHEPGREIDYITPARLDELLFSAILESHDARKEHQSFVKIMKDRGINVVELTDLVAETYDLASKAAKEEFIETFLEETVPVLTEANKKAVRAFLLSKPTHEMVEFMMSGITKYELGVESENELIVDPMPNLYFTRDPFASVGNGVTIHFMRYIVRRRETLFARFVFRNHPKLVKTPWYYDPAMKMPIEGGDVFIYNNETLVVGVSERTDLDTITLLAKNIKANKEVEFKRIVAINVPKWTNLMHLDTWLTMLDKNKFLYSPIANDVFKFWDYDLVNGGAEPQPQLNGLPLDKLLASIINKEPVLIPIGGAGATEMEIARETNFDGTNYLAIKPGLVIGYDRNEKTNAALKAAGITVLPFHGNQLSLGMGNARCMSMPLSRKDVKW

SEQ ID NO: 4 - protein, 416 aa, Mycoplasma hominis
GGSHHHHHSVFDSKFNGIHVYSEIGELETVLVHEPGREIDYITPARLDELLFSAILESHDARKEHQSFVKIMKDRGINVVELTDLVAETYDLASKAAKEEFIETFLEETVPVLTEANKKAVRAFLLSKPTHEMVEFMMSGITKYELGVESENELIVDPMPNLYFTRDPFASVGNGVTIHFMRYIVRRRETLFARFVFRNHPKLVKTPWYYDPAMKMPIEGGDVFIYNNETLVVGVSERTDLDTITLLAKNIKANKEVEFKRIVAINVPKWTNLMHLDTWLTMLDKNKFLYSPIANDVFKFWDYDLVNGGAEPQPQLNGLPLDKLLASIINKEPVLIPIGGAGATEMEIARETNFDGTNYLAIKPGLVIGYDRNEKTNAALKAAGITVLPFHGNQLSLGMGNARCMSMPLSRKDVKW

SEQ ID NO: 5 - protein, 9 aa, Artificial
  His-tag
MGGSHHHHH

(19) United States
(12) Patent Application Publication
(10) Pub. No.: US 2010/0303893 A1
(43) Pub. Date: Dec. 2, 2010

(54) NOVEL COMPOUND FOR TREATMENT OF TUMOR

(76) Inventors: Yongzhang Luo, Beijing (CN); Hao Zhou, Beijing (CN); Qingxin Lei, Beijing (CN); Guodong Chang, Beijing (CN)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/161,494

(22) PCT Filed: Jan. 19, 2007
(86) PCT No.: PCT/CN2007/000203

§ 371 Date: May 3, 2010

(30) Foreign Application Priority Data
Jan. 20, 2006 (CN) 200610011246.4

(51) Int. Cl. A61K 9/127; C12N 11/08; A61K 38/50; A61K 9/14; A61P 35/00; A61P 35/04

(52) U.S. Cl. 424/450; 435/180; 424/94.3; 424/484

(57) ABSTRACT

Specific modifying agent is coupled to an anti-tumor protein on a certain site. It conquers the disadvantages which include high antigenicity, short circulating half-life, nonuniform modified sites, inhomogeneous component, reduced activity and uncontrollable quality of the products prepared by non-specific modifying method. The anti-tumor protein coupled with specific modifying agent can be used for the treatment of tumor and for manufacturing an anti-tumor medicament.

### FIELD OF THE INVENTION

The present invention relates to methods for preparing recombinant arginine deiminase with bioactivity and metabolic stability. The present invention also provides pharmaceutical conjugates comprising the arginine deiminase, pharmaceutical compositions comprising the arginine deiminase, and kits comprising the arginine deiminase and the pharmaceutical composition. The present invention further provides the use of the arginine deiminase and the pharmaceutical composition for preventing, diagnosing, and treating tumors.

### BACKGROUND OF THE INVENTION

Hepatoma and malignant melanoma are diseases which are fatal to most of the patients within one year after diagnosis. Hepatoma is one of the most common malignant tumors in China, which develops fast and is hard to cure. The therapeutic effect is not satisfying and the patients suffering from hepatoma typically has a short life span. That is why hepatoma is called “the king of the cancers”. The data of current investigation show that the annual mortality of hepatoma in China is 20.4 persons/100,000 persons, which is 18.8% of all the malignant tumors. The mortality rose from the third place in 1970s to the second place, which is second only to lung cancer in the city and to stomach cancer in the countryside. The malignant melanoma is an acute and malignant cancer, the incidence of which increases fastest in America. Meanwhile the dermal melanocytoma is 5% of all the dermal malignant cancers, while more than 75% of the patients who die of malignant cancers are those suffering from malignant melanoma. In 2003, about 60,000 Americans have been diagnosed to be suffering from melanoma, wherein 10,000 cases are lethal. If melanoma can not be treated in the early stage, it can develop into malignant tumor and transfer to the whole body with a great efficiency, unlike the other tumors.

The methods for specifically eliminating some essential amino acids from the blood can be used for treating certain cancers. In the methods, a famous example is the treatment of acute lymphocytic leukemia by using L-asparaginase to reduce the concentration of the asparagine in blood. The commonly-used L-asparaginase is isolated from Escherichia coli. But due to the inherent antigenicity and the short circulating half life, the application of the enzyme is limited greatly (Y. K. Park et al., Anticancer Res., 1: 373-376 (1981)). The half life can be prolonged remarkedly and the antigenicity can be reduced significantly, if L-asparaginase of the Escherichia coli is modified by polyethylene glycol (Y. K. Park et al., Anticancer Res., 1: 373-376 (1981), Y. Kamisald et al., J. Pharmacol. Exp. Ther., 216: 410-414 (1981), Y. Kamisaki, Gann., 73: 470-474 (1982)). Although it is possible to treat some tumors to some extent by eliminating certain essential amino acids, the essential amino acids are also required by the growth of the normal cells. As a result, the reduction of certain essential amino acids in blood leads to some severe side effects.

It has been shown that some tumor cells have metabolic mode which is different from normal cells. The demand for certain types of amino acids by the tumor cells is also different from the normal cells. Based on the theory, it is found that some tumors, e.g., hepatoma and malignant melanoma, can be controlled with little effect on normal cells, by degrading certain non-essential amino acids, e.g. arginine. One arginine deiminase from Pseudomonas pudita can inhibit and kill the tumor cells in vitro. However, the inherent defects of the arginine deiminase of Pseudomonas pudita (e.g., activity of the enzyme is low in the condition of a neutral pH environment and it can be eliminated quickly) limit its application in the treatment of tumors. It has been confirmed that another arginine deiminase from the Mycoplasma arginini with a molecular weight of 46,000 Da is able to keep active under a neutral pH condition and inhibit the growth of the tumor in experimental animal models (Takaku et al. Int. J. Cancer, 51:244-249 (1992) and U.S. Pat. No. 5,474,928, which are incorporated herein by reference). But the arginine deiminase, as a heterologous protein from the microbe, also has the problems of high antigenicity, short circulating half life, and easy degradation in the body of the experimental animal. Some reports show the protein coupled with polyethylene glycol can prolong the half life significantly, reduce the antigenicity treat tumors (C. M. Ensor et al. Cancer Research, 62: 5443-5550 (2002)). Although coupling with polyethtlene glycol can make the arginine deiminase be used in clinical treatment of tumors (P. A. Ascierto et al. J. Clin. Oncol., 22: 1815-1822 and 23: 7660-7668 (2005)), the used coupling mode is a multiple-site and inhomogeneous modification, which leads to non-uniform shapes of modified proteins and uncontrolled preparation quality. As a result, the potency and the drug metabolism of the modified protein products of different batches are hard to evaluate, and meanwhile, the difference of clinical treatment effects of patients are hard to explain, resulting from the inherent nonuniformity of the drug, which may severely affect the formulation of the pertinent treatment protocols for different tumor patients.

Compared with chemical drugs, the polypeptide and protein drugs have the advantages of low toxicity/side effects and little drug resistance etc. In order to achieve higher activity, bioavailability, and lower degradation in vivo, the protein drugs are usually administrated intravenousely. But in this context, the half-life of low molecular weight protein drugs would be very short, not only because of the degradation, but also the quick elimination via kidney. In blood, if the proteins' hydraulic radiuses are larger than that of albumin or their molecular weights are larger than 66 kDa, they can be reserved stably in circulation. However, protein of lower molecular weight will be quickly eliminated from the blood via glomeruli. As such, in order to maintain the effective treatment concentration of low molecular weight proteins in blood, frequent intravenous administration is required. Although treatment in such a way could achieve the therapeutic effects, but it also causes inconveniences and pains to the patients and increases the cost of treatment. Meanwhile, long term administration of some drugs may cause some side effects, for instance, immunological reactions.

Arginine deiminase as a protein drug also has the disadvantages of short half life and high elimination rate in vivo. Moreover, because the arginine deiminase is from the pathogenic microorganism, it has a higher antigenicity, which may induce a strong immune response in a human body.

Using macromolecule polymers to modify proteins is a common method to change and control the dynamics characteristics of drugs, such as half-life, biological characteristics, and toxicological characteristic. The macromolecule polymer used to modify proteins should have, among others, good water-solubility, good biocompatibility, and little immunogenicity. The polyethylene glycol is a prevailing protein-modifying molecule. The polyethylene glycol has the amphipathic properties, which can be dissolved not only in water, but also in most of the organic solvents. Meanwhile, the polyethylene glycol is non-toxic, non-immunogenic, and highly soluble in water, and thus has been approved as a macromolecule polymer for drug preparation by many countries' drug administrations including SFDA of China as well as FDA of the U.S.A. Coupling the protein with macromolecule polymers, for example, the polyethylene glycol, can increase the protein's in vivo stability, decrease nonspecific adsorption and antigenicity. Once the conjugate reaches a certain molecular weight, the eliminating rate by kidney can be reduced effectively, which is an effective measure to prolong the half-life of the protein drug in vivo (Frokjaer S. et al. Nat. Rev. Drug Discov. 2005 Apr. 4(4): 298-306; Harris J M. et al. Nat. Rev. Drug Discov. 2003 Mar. 2(3): 214-21). The amino groups initially used as the reaction site in the modification of the polyethylene glycol were mainly α-amino group of N-terminus of the protein, and ε-amino group of the side chain of the lysine residue. The product of the reaction is a protein molecule non-specifically coupled with one or multiple PEG molecules. The modification of ε-amino group in the side chain of the lysine residue may generate modified isomerides due to the non-specific reaction sites.

Recently, aiming at the difference of isoelectric points between α-amino group of N-terminus of the protein and ε-amino group of the side chain of the lysine residue, polyethylene glycol modifying agents have been developed which specifically modify N-terminus of the protein and as a result, it is possible to obtain uniform modification products with modification at the identical site. Another protein site for modification with the polyethylene glycol is the mercapto group of the cysteine residue. Generally, the number of the mercapto groups is fewer than that of the amino groups in a protein, and thus modification of the mercapto groups is more specific. Using genetic engineering techniques, now it is possible to introduce a cysteine at any position of a protein to serve as a specific modification site. But introducing a cysteine as the modification site also has certain limitations because, for those proteins or polypeptides that do not contain cysteine residues, this may cause the crosslinking between the molecules, resulting in the loss of activity, and for those proteins that already contain cysteine residues, this may cause mispairing of disulfide bonds, resulting that such proteins cannot renature. In addition, the carboxyl group of the protein is also frequently used as a site for modification (Veronese F M et al. Drug Discov. Today. 2005 Nov. 1; 10(21):1451-8.). Modification techniques with polyethylene glycol have been successfully used in a plurality of protein drugs, including PEG-asparaginase (Graham M L Adv. Drug Deliv. Rev. 55, 1293-1302 Avramis Vassilios I. et al. WO9939732 and U.S. Pat. No. 6,689,762), PEG-adenosine deaminase (Levy Y et al. J. Pediatr. 113, 312-317; Davis S et al. ClinExp. Immunol. 46: 649-652; Hershfield M S et al. N Engl J Med 316: 589-596), PEG-interferons such as PEG-interferon α2a and PEG-interferon α2b etc. (Bailon P et al. C. Bioconjug. Chem. 12, 195-202, Wang Y S et al. Adv. Drug Deliv. Rev. 54, 547-570, Meng Xiantai et al. WO2005077421, Van Vlasselaer Peter et al. WO2004076474, Ballon Pascal Sebastlan et al. US2004030101, Karasiewicz Robert et al. EP0593868) etc.

Other representative macromolecule modifying agents include glucan, the polysucrose, starch, polyalanine, copolymer of adipic acid and the malonic acid, carboxymethyl cellulose, polyvinylpyrrolidone, poly 1,3-dioxolane, ethylene and maleic hydrazide copolymer, poly sialic acid, cyclodextrin, etc.

Another method of prolonging the half-life of a protein drug is to couple or fuse it with a blood protein or a fragment thereof used as a carrier to increase the molecular weight of the protein drug. For example, Fc fragment of immunoglobulin may be coupled with the target protein to prolong the half-life of the latter. For example, this strategy has been used in the Notch 1 receptor protein (Kitajewsky Jan et al. WO2005111072), erythropoietin (EPO) (Gillies Stephen D et al. WO2005063808), human somatropin (Kim Young Min et al. WO2005047337), etc. The plasma albumin is another commonly-used coupling carrier, which is used in the proteins such as some antibiotics, anti-inflammatory agents, and anti-oxidants (Ezrin Alan M et al. WO0117568, Otagiri Masaki et al. EP1571212), etc.

In addition to directly coupling the protein drug with some blood protein carriers in vitro, it is also possible to modify the drug protein in vitro to provide the drug protein with chemical reaction activity or high affinity for some molecules in vivo, so that the drug protein can react with some components of the blood upon entering into the body to form macromolecules or compounds with longer half-life. One example is the antiviral small peptide anti-RSV modified with an active group carrying maleimide, which can react with the mercapto groups of the blood proteins or the cell surface proteins (Bridon Dominique P et al. WO0069902). Another example is to introduce fatty acids to the amino acid residues on the surface of the protein by acylation reaction so as to increase the affinity of the protein to the albumin in vivo. Upon adminstered into the blood, the protein can form a larger conjugate with albumin and thereby the half-life of the protein is prolonged. The method has been used to produce long-acting insulins (Kurtzhals P et al. Biochem. J. (1995) 312, 725-731; Frokjaer S et al. Nat Rev Drug Discov. 2005 Apr.; 4(4):298-306).

Sustained release formulation is another approach of prolonging the in vivo half-life of a protein drug. The protein drug is placed in a pharmaceutical carrier, which may be a chemical macromolecule, or a physical device capable of releasing proteins slowly and continuously, to allow the protein to be released from the carrier slowly, and thereby a stable in vivo drug concentration is maintained. The commonly used sustained release formulations comprise hydrogel, microcapsule, microballoon, liposome, micro-osmotic pump, etc. (Peppas N A et al. Eur. J. Pharm. Biopharm. 50, 27-46 (2000); Packhaeuser C B et al. Eur. J. Pharm. Biopharm. 58, 445-455 (2004); Metselaar J M et al. Mini Rev. Med. Chem. 4, 319-329 (2002)). Liposome is an ultramicroscopic particle in the form of a hollow sphere with a bilayer membrane structure. The bilayer membrane is composed of amphipathic molecules, most of which are phosphatides, and forms a hydrophilic inner chamber. The hydrophilic protein drug is encapsulated in the inner chamber of the liposome and thus can be slowly released in vivo to maintain the concentration of the protein in blood and prolong the half-life. Examples are nerve growth factor (Hou Xinpu et al. CN1616087) and hemoglobin (Farmer Martha C et al. U.S. Pat. No. 4,911,929) etc. A micro osmotic pump is a physical device to control the content to be released slowly by using the difference between the exterior osmotic pressure and the interior osmotic pressure of the semi permeable membrane, which has been widely used in studying the slow-release of a variety of chemical and biological drugs in experimental animal models.

The main object of the present invention is to reduce the antigenicity of the arginine deiminase, to improve the metabolic characteristics of the protein in vivo, to make the protein be provided with higher stability and a longer half life in vivo, so that the protein can be used in clinical treatment of tumors, and the economic burden as well as the physiological pain of patients may be decreased.

### SUMMARY OF THE INVENTION

The present invention provides a conjugate formed by a modifying agent and arginine deiminase, wherein the modifying agent is capable of prolonging the half-life of arginine deiminase. The modifying agent used in the present invention may be attached to arginine deiminase via a covalent bond. These modifying agents may be selected from macromolecules, protein molecules or fragments thereof, peptides, small molecules or other compounds. The macromolecules that can be used in the present invention include, for example, polyethylene glycol with an average molecular weight in the range from 5,000 to 100,000 Daltons, preferably 5,000 to 60,000 Daltons, more preferably 5,000 to 40,000 Daltons, and most preferably 20,000 to 40,000 Daltons.

In one embodiment of the present invention, the conjugate is an arginine deiminase molecule coupled with a polyethylene glycol molecule.

In the conjugate of the present invention formed by polyethylene glycol and arginine deiminase, the coupling site is one selected from the group consisting of the α-amino group of the N-terminal amino acid residue, ε-amino group of the side chain of a lysine residue, mercapto group of the side chain of a cysteine residue, carboxyl group of the side chain of an aspartate residue, carboxyl group of the side chain of a glutamate residue, the hydroxyl group of the side chain of a tyrosine residue, the hydroxyl group of the side chain of a serine residue, and the hydroxyl group of the side chain of a threonine residue in arginine deiminase or combination thereof. Preferably, the coupling site is the α-amino group of the N-terminal amino acid residue of arginine deiminase.

In one embodiment of the present invention, the conjugate is an arginine deiminase molecule coupled with a polyethylene glycol molecule, and the coupling site is the α-amino group of the N-terminal amino acid residue of arginine deiminase.

In another embodiment of the present invention, the conjugate is an arginine deiminase coupled with a polyethylene glycol molecule at mercapto groups in additional cysteine residues, wherein the additional cysteine residues are introduced by adding cysteine residues, or peptide chains containing cysteine residues, to the N-terminus, C-terminus, or the internal region of said arginine deiminase molecule.

In another embodiment of the present invention, the conjugate is an arginine deiminase molecule coupled with a polyethylene glycol molecule at a specific site which is the carboxyl group in the aspartate or glutamate residues in said arginine deiminase molecule.

In another embodiment of the present invention, the conjugate is an arginine deiminase molecule coupled with a polyethylene glycol molecule at a specific site which is the hydroxyl group in the tyrosine, serine or threonine residues in said arginine deiminase molecule.

In one embodiment of the present invention, the modified site of the arginine deiminase in the conjugate is unique and definite.

Proteins that can be used in the conjugate of the present invention are selected from a group consisting of albumin, immunoglobulin, thyroxin-binding protein, transthyretin, transferrin, fibrinogen and fragments thereof.

In one embodiment of the present invention, the conjugate includes an arginine deiminase coupled to albumin(s). The conjugate is formed by coupling one arginine deiminase molecule to one or more albumins. The conjugate may be obtained via chemical modification, fusion expression, or other methods. Preferably, the albumin is human serum albumin or its fragment. In another embodiment of the present invention, the conjugate includes an arginine deiminase coupled to immunoglobulin Fc fragment(s). The conjugate is formed by coupling one arginine deiminase molecule to one or more immunoglobulin Fc fragments. The conjugate may be obtained either via chemical modification, or via fusion expression, or via other methods. Preferably, the Fc fragment is a fragment of the Fc region of human immunoglobulin IgG.

The conjugate of the present invention also includes arginine deiminase modified with small molecules or small peptides or other compounds, wherein the modified arginine deiminase is capable of reacting or binding with other molecules or components in vivo, allowing modified arginine deiminase to form larger conjugate with other molecules or components in vivo. The modified arginine deiminase contains a reactive group capable of forming covalent bond with an amino, hydroxyl, or mercapto group of a blood component. The reactive group can be, for example, maleimide, which may react with blood components such as the mercapto group of albumin. In this way, the conjugate of the present invention will have strong affinity to some blood components such as albumin or immunoglobulin, allowing the formation of a larger conjugate.

The conjugate of the present invention also includes arginine deiminase modified with other molecules or small peptides. For example, the arginine deiminase can be a glycosylated, phosphorylated or acylated product, in which modifying sites are amino acid residues existing in the original protein sequence or amino acid residues generated by mutation.

The conjugate of the present invention formed by a modifying agent and arginine deiminase may also be a conjugate formed through non-covalent bonds between the arginine deiminase and other carriers which comprises proteins, small molecules or other substances functioning as carriers.

The conjugate of the present invention may form a sustained-release formulation with a bio-compatible carrier. The sustained-release formulation may be in a form selected from the group consisting of microcapsule, hydrogel, microsphere, micro-osmotic pump or liposome.

The arginine deiminase used in the conjugate or the sustained-release formulation is derived from Mycoplasma hominis, Mycoplasma arthritidis, or Mycoplasma arginini, or the arginine deiminase is an arginine deiminase of Mycoplasma hominis, Mycoplasma arthritidis or Mycoplasma arginini prepared by cloning via genetic recombination technology.

In one specific embodiment of the present invention, the arginine deiminase contained in the conjugate or sustained-release formulation is preferably from Mycoplasma hominis, the wild type of which has a sequence as shown in SEQ ID No.1. More preferably, the arginine deiminase contained in the conjugate or sustained-release formulation of the present invention is E. coli expressed wild-type recombinant Mycoplasma hominis arginine deiminase, which has the sequence as shown in SEQ ID No.1 or SEQ ID No.2.

In the conjugate or sustained-release formulation of the present invention, the arginine deiminase may be an active fragment, mutant, derivative, isomer of arginine deiminase or a combination thereof, preferably an active fragment, mutant, derivative, isomer of arginine deiminase from Mycoplasma or a combination thereof, and more preferably an active fragment, mutant, derivative, isomer of arginine deiminase from Mycoplasma hominis or a combination thereof. For example, in the conjugate of the present invention, the derivative of arginine deiminase is a sequence with an additive peptide of 1-15 amino acid residues in length at the N-terminus or C-terminus, preferably with an additive His-tag containing peptide MGGSHHHHH (SEQ ID No.5) at the N-terminus, having a sequence as shown in SEQ ID No.3 or SEQ ID No.4.

The present invention also provides a pharmaceutical composition comprising a conjugate or sustained-release formulation of the present invention and a pharmaceutically acceptable carrier.

The present invention also provides kits comprising the conjugate, composition or sustained-release formulation of the present invention and instruction for the usage thereof.

The present invention also provides method of preparing an arginine deiminase-containing conjugate as mentioned above comprising the steps of mixing a macromolecule polymer with arginine deiminase and allowing reaction under proper conditions including solution, temperature, pH, and reaction molar ratio. Preferably, the pH used in the method is pH 3 to pH 10. In the method mentioned above, the conjugate product may be purified, for example, via ion exchange column or gel filtration.

The present invention also provides the use of an arginine deiminase-containing conjugate, composition, sustained-release formulation, or kit as mentioned above in the prevention, diagnosis and treatment of tumors and in the prevention, diagnosis or treatment of other arginine-related diseases.

The present invention also provides a method for prolonging the in vivo half-life of arginine deiminase comprising the step of forming a conjugate between arginine deiminase and a modifying agent, or comprising the step of providing a sustained-release formulation comprising arginine deiminase or arginine deiminase containing conjugate and a bio-compatible substance.

These and other aspects of the present invention will be elucidated in the following detailed description of the invention. It should be understood that the above general description and the following detailed description as well as the examples are merely for the purpose of illustration, and are not intended to limit the scope of present invention.

### BRIEF DESCRIPTION OF THE DRAWINGS

FIG. 1 depicts the amino acid sequence of a wild-type arginine deiminase from Mycoplasma hominis (SEQ ID No.1).

FIG. 2 depicts the amino acid sequence of a wild-type Mycoplasma hominis arginine deiminase expressed by E. coli, yeast or mammalian cells (SEQ ID No.2).

FIG. 3 depicts the amino acid sequence of Mycoplasma hominis arginine deiminase derivative expressed by E. coli (SEQ ID No.3).

FIG. 4 depicts the amino acid sequence of Mycoplasma hominis arginine deiminase derivative expressed by E. coli, yeast or mammalian cells (SEQ ID No.4).

FIG. 5 is a graph showing the low antigenicity of Arginine Deiminase (ADI) coupled to polyethylene glycol at a single site of N-terminus in experimental animal. 9 healthy Kunming mice with an average body weight of around 25 g were devided into 3 groups, tail-vein injected with arginine deiminase, arginine deiminase specifically modified with 20 kDa polyethylene glycol at a single site of the N-terminus and arginine deiminase non-specifically modified with polyethylene glycol at multi-sites respectively; the dosage is set 15 mg/kg body weight.

FIG. 6 is a graph showing that the arginine deiminase specifically modified with polyethylene glycol at a single site of the N-terminus preserves its integral biochemical activity. Arginine deiminase was dissolved in 100 μl phosphate buffered saline (PBS) to a final concentration of 10 μg/ml, and then L-arginine as the substrate of the arginine deiminase was added to a final concentration of 10 μmol/L. The reaction mixture system was incubated in 37° water bath for 10 minutes, and then the mixture was taken out to measure the change of L-arginine concentration using an assay kit following the instruction of the manufacturer.

FIG. 7 is a graph showing that the arginine deiminase specifically modified with polyethylene glycol at a single site of the N-terminus preserves its intact activity of inhibiting tumor cell proliferation. The mice malignant melanoma cells (B16/F10) were cultured in DMEM (10% serum added) until logarithmic phase. And then the cells were starved under serum-deprived condition for 12 hours. Normal culture media containing 10% fetal bovine serum and dual antibiotics were added to the tumor cells. Treatment groups were treated with arginine deiminase and modified arginine deiminase (including arginine deiminase specifically modified at a single site and arginine deiminase non-specifically modified at multi-sites) respectively to a final concentration of 10 μg/ml; and the control group is added with an equal volume of normal saline.

FIG. 8 depicts the activity of the arginine deiminase specifically modified with polyethylene glycol at a single site of the N-terminus in treating mouse tumor models. C57 mice with an average body weight of about 20 g were given subaxillary inoculation with B16/F10 malignant melanoma cells, 2×10⁶ cells per mouse. The mice were randomly grouped the next day, 8 mice per group. Designated were the negative control group (normal saline), the positive control group (arginine deiminase 5 mg/kg body weight (1.7 U/mouse), daily administration), and the treatment groups were employed to treatment of arginine deiminase specifically modified at a single site and arginine deiminase non-specifically modified at multi-sites, with an administration interval of once every 3 days and once every 7 days, respectively. A, tail-vein administration group; B, subcutaneous administration group.

FIG. 9 is a graph showing that the arginine deiminase specifically modified with polyethylene glycol at a single site of the N-terminus significantly prolonged the survival time of tumor-bearing mice. C57 mice with an average body weight of about 20 g were given subaxillary inoculation with B16/F10 malignant melanoma cells, 2×10⁶ cells each mouse. The mice were randomly grouped the next day, 8 mice per group. Designated were the negative control (normal saline), the positive control (chemical tumor-inhibition drug, daily administration), the treatment group using arginine deiminase specifically modified at a single site (the interval of treatment is set once every 7 days), respectively.

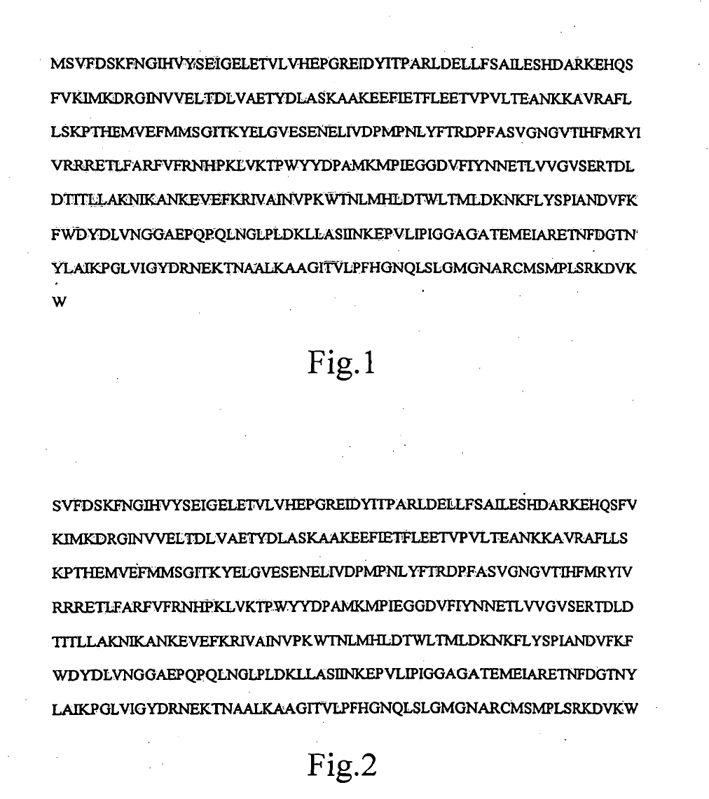

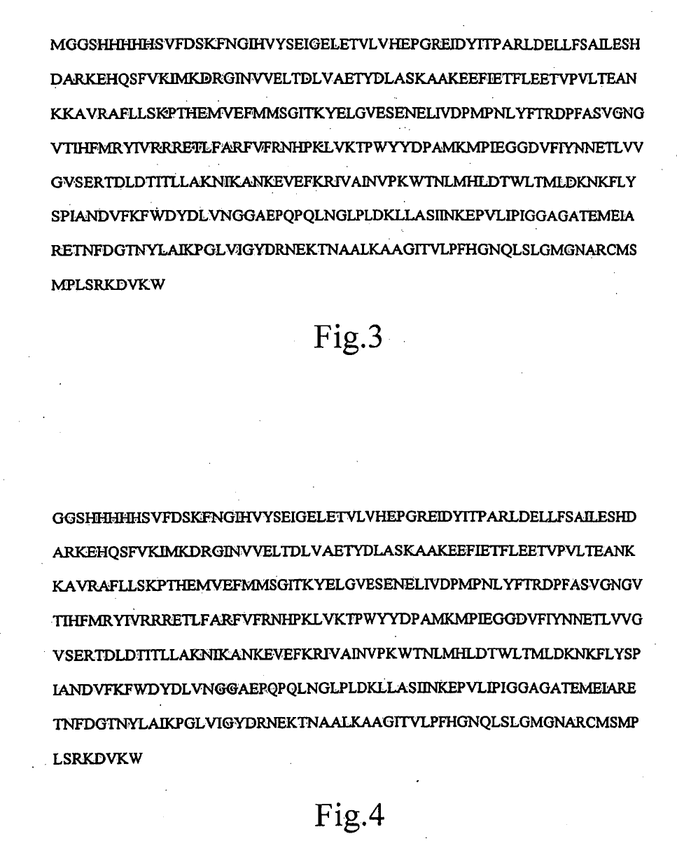

### DETAILED DESCRIPTION OF THE INVENTION

The arginine deiminase of the invention can be derived from any microorganisms possessing arginine deiminase, unless otherwise indicated. Preferably, the arginine deiminase is wild type Mycoplasma hominis, as shown in SEQ ID No.1. The arginine deiminase of the invention include wild type arginine deiminase (i.e., naturally existing form) or the mutant, fragment, isomer or derivative thereof having activity or their combinations. The arginine deiminase can be fermented and purified from E. coli (as shown in SEQ ID No.1 and SEQ ID No. 2), expressed in animal cells or fermented from yeast (as shown in SEQ ID No.2). Among them, the amino acid sequence of wild type Mycoplasma hominis arginine deiminase expressed in animal cells is shown in SEQ ID No.2; the amino acid sequence of recombinant Mycoplasma hominis arginine deiminase expressed in Yeast has the sequence as shown in SEQ ID No.2 or has a insertion or deletion of less than 10 amino acids at the N terminus of this sequence; and the recombinant Mycoplasma hominis expressed in E. coli has the sequence as shown in SEQ ID No. 1 or/and SEQ ID No. 2.

The invention provides a kind of arginine deiminase product with low antigenicity and a long half life. Compared with the unmodified arginine deiminase, it not only keeps its activity in inhibiting tumor cell growth, but also has very low antigenicity, higher stability in vivo and longer half life in vivo.

Polyethylene glycol or “PEG” refers to a mixture of branced or linear condensation polymers of ethylene oxide and water, represented by the general formula H(OCH₂CH₂)ₙOH wherein n is at least 4. “Polyethylene glycol” or “PEG” is used in combination with a numeric suffix to indicate the approximate average molecular weight thereof. For example, PEG 20,000 refers to polyethylene glycol having an average molecular weight of about 20,000 Daltons; PEG 40,000 refers to polyethylene glycol having an average molecular weight of about 40,000 Daltons.

This invention is based on a surprising discovery: arginine deiminase which is specifically modified with PEG at a single site also has significant effects in treating certain tumors and preventing tumor metastasis. Compared with multi-site modified arginine deiminase and arginine deiminase modified non-specifically at a single site, the arginine deiminase modified non-specifically at a single site not only preserves its low antigenicity, good tumor inhibiting activity but also possesses higher activity, better homogeneity, and the reproducity of the product quality of different batches remarkedly superior to non-specifically modified arginine deiminase, and thus can be utilized in clinical tumor treatment and the preparation of anti-tumor medicaments.

In the present invention, the gene encoding arginine deiminase may be obtained, cloned or produced from any source, for example, microorganisms, recombinant biotechnology or any combination thereof. Preferably, arginine deiminase is cloned from microorganisms of Mycoplasma. More preferably, the arginine deiminase is cloned from Mycoplasma arginini, Mycoplasma hominis, Mycoplasma arthritides, or any combination thereof. In particular, the arginine deiminase used in the present invention may have one or more of the amino acid sequences as shown in SEQ ID No. 1.

A mutant of arginine deiminase refers to a protein molecule obtained by the replacement, deletion, insertion of amino acids. A fragment of arginine deiminase refers to a sequence belonging to any smaller part of SEQ ID No.1. The sequence can be obtained via enzyme cleavage, expressed by genentic engineering or obtained by polypeptide synthesis. An isomer of arginine deiminase refers to a molecule with the same amino acid sequence as the wild-type arginine deiminase but with different conformations, including difference in the secondary or tertiary protein structure, or different optical activity in regional amino acids. The isomer may be a naturally occurring mutant or obtained via artificial design. A derivative of arginine deiminase refers to a product generated by modification of the wild-type arginine deiminase. Modification refers to covalently attaching one or more small molecules, such as phosphoric acid molecules or carbohydrate molecules, or oligopeptide with less than 20 amino acids, to the protein at any amino acid of the protein. In a preferred embodiment, the derivative may be an arginine deiminase modified at N-termina with an addition of peptide containing a His-tag which may be consisted of 3 to 10 amino acids. The derivative preferably has the sequence shown in SEQ ID No.3 or SEQ ID No.4 and includes derivatives sequences on the sequence shown in SEQ ID No.3 or SEQ ID No.4. The combination of the active mutants, fragments, isomers or derivatives of arginine deiminase means that the product simultaneously has two or more of the modifications mentioned above, for example, but not limited to, a mutant of a fragment or a modified mutant, etc.

The invention provides a kind of arginine deiminase product having a longer half life. This product is consisted of arginine deiminase and a non-arginine deiminase modifying component. The arginine deiminase is defined as above, and the modifying component can be any form, including, but not limited to macromolecule polymers, protein molecules, peptides or chemicals in any other forms. Arginine deiminase and the modifying component are linked by either covalent bonds or non-covalent interaction to form a stable conjugate or a stable composition. This product has the biological activity of arginine deiminase and has a longer half life in vivo and much lower antigenicity than the unmodified arginine deiminase. Therefore, it can serve as an anti-tumor drug.

In one embodiment of the invention, it provides a conjugate of a modifying agent and arginine deiminase, namely, a modified arginine deiminase. The term “conjugate” used herein refers to a modified arginine deiminase. Modification refers to linking one or more modifying agents by covalent bonds directly or indirectly to arginine deiminase. The modifying agent can be a polymer or a protein or a fragment thereof that is biocompatible and can increase the half life of arginine deiminase in blood. The modifying agent can be either chemically coupled to arginine deiminase or linked to the arginine deiminase via fusion expression.

The macromolecule polymers as mentioned above refer to non-peptide macromolecule polymers, which can either have its own bioactivity or not. The suitable polymers include, but not limited to, polyenol compounds, polyether compounds, polyvinylpyrrolidone, poly amino acids, copolymer of divinyl ether and maleic anhydride, N-(2-hydroxypropyl)-methacrylamide, polysaccharide, polyoxyethylated polyol, heparin or its fragment, poly-alkyl-ethylene glycol and its derivatives, copolymers of poly-alkyl-ethylene glycol and its derivatives, poly(vinyl ethyl ether), a,P-Poly[(2-hydroxyethyl)-DL-aspartamide], polycarboxylates, poly oxyethylene-oxymethylenes, polyacryloyl morpholines, copolymer of amino compounds and oxyolefin, poly hyaluronic acid, polyoxiranes, copolymer of ethanedioic acid and malonic acid, poly (1,3-dioxolane), ethylene and maleic hydrazide copolymer, poly sialic acid, cyclodextrin, etc. Preferably, the polymer is polyethylene glycol.

The polyenol compounds as mentioned above include, but not limited to, polyethylene glycol (including monomethoxy polyethylene glycol, monohydroxyl polyethylene glycol), polyvinyl alcohol, polyallyl alcohol, polybutenol and the like, and their derivatives, such as lipids.

The polyether compounds include, but not limited to poly alkylene glycol (HO((CH2)ₓO)ₙH), polypropylene glycol, polyoxyrehylene(HO((CH₂)₂O)ₙH), polyvinyl alcohol((CH₂CHOH)ₙ).

The poly amino acids as mentioned above include, but not limited to, polymers of one type of amino acid, copolymers of two or more types of amino acids, for example, polyalanine.

The polysaccharides as mentioned above include, but not limited to, glucosan and its derivatives, for example dextran sulfate, cellulose and its derivatives (including methyl cellulose and carboxymethyl cellulose), starch and its derivatives, polysucrose, etc.

In one specific embodiment of the present invention, arginine deiminase is coupled with proteins or peptides, wherein one or more proteins or peptides are directly or indirectly linked to arginine deiminase. The proteins can either be naturally existing proteins or their fragments, preferably but not limited to naturally existing human serum proteins or their fragments, including but not limited to thyroxine-binding protein, transthyretin, a1-acid glycoprotein, transferrin, fibrinogen, immunoglobulin, albumin and their fragments. ‘Fragments’ of a protein refer to any part of the protein that is smaller than the whole protein but retains the function of the protein as a carrier. Arginine deiminase is directly or indirectly linked to the carrier protein via a covalent bond. Direct linking means that one amino acid of arginine deiminase is directly linked to one amino acid of the carrier protein, via peptide bond or a disulfide bridge. Indirect linking refers to the linkages between arginine deiminase and carrier proteins, via originally existing chemical groups therebetween or specific chemical groups added through biological or chemical ways, or the combination of the abovementioned linkages.

In one embodiment of the present invention, the polyethylene glycol (PEG)-modified arginine deiminase is characterized in that one arginine deiminase molecule is covalently coupled to one PEG molecule. The coupling site of arginine deiminase may be one of the N-terminal α-amino group, the ε-amino group on the side chain of a lysine residue, the mercapto group of a cysteine residue, the carboxyl group on the side chain of an aspartate residue, the carboxyl group on the side chain of a glutamate residue, preferably but not limited to the N-terminal α-amino group. All the polyethylene glycol molucules used for coupling may be a linear or branched molecule, with a molecular weight of 1,000 to 100,000 Daltons, preferably but not limited to 5,000 to 40,000 Daltons, more preferably but not limited to 20,000 to 40,000 Daltons. It is preferred to use the N-terminal α-amino group of a PEG specifically modified arginine deiminase to form a product modified at a single site.

In one embodiment of the present invention, it involves the chemically reactive modified arginine deiminase which have chemically reactive groups and the ability to react with certain substances in blood and form a stable covalent bond in vivo. Such substances include, without limitation, molecules with one reactive group which can react and form a covalent bond with an amino group, a hydroxyl group, a mercapto and the like in blood. The active group is prefererally but not limited to maleimide which can react with the mercapto of blood proteins including but not limited to movable blood proteins e.g. albumin.

In one specific embodiment of the present invention, the conjugate comprising arginine deiminase is a conjugate molecule with a specific composition, which is formed via non-covalent interaction between arginine deiminase and other molecules. This conjugate has the activity to inhibit tumor growth and has longer in vivo half-life than arginine deiminase.

Another embodiment of the present invention relates to a sustained-release composition comprising arginine deiminase. The sustained-release composition is a stable composition consisting of arginine deiminase or the conjugate thereof and a pharmaceutical carrier. Arginine deiminase in this composition still has biological activity, and at the same time, its in vivo half-life is prolonged because of the carrier which changes the pharmacokinetic characteristics of the drug. It is preferred without limitation that the chemical or physical substained-release substances are used in the in vivo sustained-release technology. In a preferred embodiment, arginine deiminase or the conjugate, the composition thereof is embedded in a liposome. In another preferred embodiment, arginine deiminase or the conjugate, the composition thereof is contained in a micro-osmotic pump.

The present invention also provides a pharmaceutical composition which contains the arginine deiminase or its conjugate or composition. The pharmaceutical composition is composed of arginine deiminase-containing pharmaceutical composition and a proper pharmaceutical carrier. The pharmaceutical carriers used in this invention include carriers, excipients, or stabilizers, which are nontoxic to the cells or mammals to be contacted at selected doses and concentrations. A commonly used physiologically acceptable carrier is an aqueous pH buffer solution. Examples of physiologically acceptable carriers include solutions such as phosphate buffer solution, citrate buffer solution and other organic acid buffer solution; anti-oxidants including ascorbic acid; polypeptides with low molecular weight (no more than 10 residues); proteins such as serum albumin, glutin or immunoglobulin; hydrophilic polymers such as polyvinylpyrrolidone; amino acids such as glycine, glutamine, asparagine, arginine or lysine; monosaccharide, disaccharide and other carbohydrates including glucose, mannose or dextrin; chelating agents such as EDTA; sugar alcohols such as mannitol or sorbitol; salt-forming counter ions such as sodium ion; and/or nonionic surfactants such as TWEEN®, PEG and PLURONICS®.

The aforementioned pharmaceutical composition of arginine deiminase or its conjugate generally can be combined with pharmaceutical carriers (excipients) to form a pharmaceutical composition. The pharmaceutical carriers can comprise one or more physiologically acceptable compounds which can be used to stabilize such composition, or increase or decrease the absorption of active reagents. Physiologically acceptable compounds include, for example, carbohydrates such as glucose, sucrose or dextran; antioxidants such as antiscorbic acid or glutathione, chelating agents, proteins with a low molecular weight, protecting and absorption enhancers such as lipids, components which can decrease the elimination or hydrolysis of active reagents, excipients or other stabilizers and/or buffers.

Other physiologically acceptable compounds include wetting agents, emulsifying agents, dispersing agents, or protecting agents which are especially useful in preventing the growth or influences of microorganisms. Various protecting agents are well-known including phenol and antiscorbic acid. A person skilled in the art is able to expect that the selection of pharmaceutical carriers, which comprise physiologically acceptable compounds, depends on, e.g. the administration route and specific physiological-chemical characteristics of the active reagents.

The preferred excipients are sterilized and usually free of dys-materials. These compounds can be sterilized through routine and well-known sterilizing techniques.

The present invention also provides a kit which includes the aforementioned arginine deiminase or its conjugate, composition and the operating instruction.

The present invention also involves methods for preparing the aforementioned conjugate or composition of arginine deiminase. In particular, the present invention relates to methods for preparing a conjugate of arginine deiminase which is coupled by PEG at a single site of N-terminus.

The present invention also provides methods of the prophylaxis, diagnosis or treatment of cancers and other arginine-related diseases as well as the inhibition of tumor growth and metastasis, wherein the conjugates or compositions of arginine deiminase with low antigenicity, high activity and long half life are included without limitation as drugs. The tumors that are suitable to be treated with the method include, but not limited to, lung cancer, hepatoma, gastric cancer, esophageal cancer, bone cancer, pancreatic cancer, lymphoma, colon cancer, breast cancer, prostate cancer, oral cancer, nasopharyngeal carcinoma, uterine cervix cancer, leukemia, malignant melanoma, sarcoma, renal cancer, biliary cancer, etc. It is preferred without limitation that a conjugate of arginine deiminase which is coupled with PEG at a single site of N-terminus is used to treat tumors.

The present invention also provides the administration routes of the aforementioned conjugate or composition of arginine deiminase or both in the prophylaxis, diagnosis or treatment of tumors and other arginine-related diseases. The administration routes include, but not limited to, intravenous injection, intravenous drip, venous canal administration, arterial canal administration, intramuscular injection, intraperitoneal injection, oral administration, inhalation administration, subcutaneous administration, dermal administration, rectal administration, vaginal administration, nasal mucosa administration, oral mucosa administration, ocular administration, or other administration routes.

The present invention also provides a method and use of the abovementioned conjugate or composition of composition of arginine deiminase or both in the preparation of anti-tumor medicaments.

PEG is directedly conjugated to the N-terminal amino acid residues of arginine deiminase, and the single modified protein product is purified. The single-site specifically modified arginine deiminase has a similar low antigenicity as multiple-site non-specifically modified arginine deiminase, which hardly induces immune responses in experimental animals; meanwhile, the single-site modified arginine deiminase has a higher activity than the multiple-site modified arginine deiminase at the same molar concentration; the single-site specifically modified arginine deiminase exhibits a higher activity in inhibiting tumor activity than multiple-site modified arginine deiminase in parallel treatments of mice suffering from tumors; the single-site specifically modified arginine deiminase exhibits a much better homogenicity and unicity of materials than multiple-site modified arginine deiminase in HPLC; the method for preparing single-site specifically modified arginine deiminase has a higher utilization rate of modifying materials, that is to say, fewer macromolecule materials are capable of producing more modified products.

It has been demonstrated that the conjugate of arginine deiminase modified by PEG at a single site of N-terminus has a higher activity in inhibiting tumor cell proliferation and mice tumor growth and a higher stability and enzyme activity than multiple-site modified arginine deiminase and single-site non-specifically modified arginine deiminase with the same dosage; moreover, the former has a better component homogenicity, purity, structural homogenicity and reproducibility of product quality of different batches. Additionally, the modified product of arginine deiminase modified by PEG at a single-site of N-terminus can effectively reduce antigenicity and slow down the metabolism of arginine deiminase and thus increase the half life of arginine deiminase in in vivo pharmacokinetic experiments. In a preferred embodiment, the N-terminal PEG single-site modified arginine deiminase conjugate can be used for tumor treatment and for the preparation of anti-tumor medicaments.

At the same time, compared with the PEG non-directedly conjugated arginine deiminase, or PEG directedly conjugated other types of proteins, the N-terminus directedly conjugated arginine deiminase has increased for several times in preparation efficiency, enzyme activity; the dosage of the directedly conjugated arginine deiminase is much less to achieve the same level of tumor inhibition; although each of the arginine deiminase is modified with one PEG molecule, compared with the arginine deiminase modified by a plurity of PEG molecules with the same molecular weight, the former has a comparable effect in decreasing antigenicity and increasing half life.

In Poly(ethylene glycol) (PEG) conjugated arginine deiminase: effects of PEG formulations on its pharmacological properties Journal of Controlled Release, 80: 259-271 (2002), F. W. Holtsberg and his colleagues introduced the method for modification of arginine deiminase with PEG. It is described that, under a standard reaction condition, the molar ratio of PEG reagent to arginine deiminase of 40:1 was used for all PEGs≦20 kDa and a molar ratio of 50:1 was used for all PEGs≧30 kDa, and the reaction efficiency was typically ≧95%. In our experiments, a molar ratio of PEG reagent to arginine deiminase of 2:1 was used, the reaction efficiency commonly reached to 70%, so the preparation efficiency has increased by 10 times or more. Compared with other single PEG modified proteins, such as Interferon-β1b, Bioconjugate Chem. 2006, 17, 618-630, the reaction efficiency has increased by approximately 5 times.

In the same article, F. W. Holtsberg et al have mentioned that, under general reaction conditions, the enzyme activity of multiple PEG modified arginine deiminase decreased by 40% to 65%, while the single PEG modified arginine deiminase has the same level of enzymatic activity as before modification, which is twice that of the multiple PEG modified product.

In C. M. Ensor et al, Cancer Research, 62: 5443-5450 (2002), Mike A. Clark et al, WO9851784, and Mike A. Clark et al, WO2002044360, the dosage of multiple PEG modified arginine deiminase is 5 IU/mouse/week in mice tumor models, however, in our experiments, the dosage of 1.7 IU/mouse/week also achieved a prominent tumor inhibition. It can be seen that the dosage in our experiments had decreased to ⅓of the previously disclosed dosage.

The single PEG modified arginine deiminase of the present invention has a comparable level of decrease in antigenicity to the multiple PEG modified arginine deiminase with the same molecular weight as reported in F. W. Holtsberg et al. Journal of Controlled Release, 80: 259-271 (2002), Mike A. Clark et al, WO9851784, and Ascierto et al, Journal Of Clinical Oncology, 23:7660-7668 (2005); and in the respect of prolonging half life, weekly use of the single PEG modified arginine deiminase also achieved prominent treatement efficacy.

Other single PEG modified proteins, such as Interferon-β1b, Bioconjugate Chem. 2006, 17, 618-630 have a half life increased about 10 times, and the single PEG modified arginine deiminase of the present invention has a half life increased by approximately 20 times.

### EXAMPLES

### Example 1

### Coupling of PEG to the N-Terminus of Arginine Deiminase

The recombinant arginine deiminase (Protgen Ltd.) was dialyzed into 10 mM phosphate buffered saline, pH 7.0. Protein concentration was determined by measuring absorbance at 280 nm using UV spectrophotometer (Agilent Technologies), and then was adjusted to 4 mg/ml. When coupling with 20 kDa or 40 kDa PEG, 40 mg of 20 kD PEG (mPEG-ButyrALD 20 kDa, Nektar) solid or 80 mg of 40 kD PEG (mPEG-ButyrALD 40 kDa, Nektar) solid was added to 10 ml protein solution (containing 40 mg protein), and the mixture was stirred at room temperature until PEG solid dissolved completely and the molar ratio of PEG and arginine deiminase was 2:1. CH₃BNNa (Sigma) was added as reductant to achieve a final concentration of 20 mM, and the pH value of the solution was adjusted to 7. After resting at room temperature for 10 hours, most of the arginine deiminase was modified with mono-PEGylation, and a small amount of arginine deiminase was modified at multi-sites. The solution can be purified directly through column chromatography after being diluted to reduce ionic strength, or stored at 4° C. for short-term storage after being diluted 10-fold.

### Example 2

### Purification of Arginine Deiminase Modified with PEG at a Single Site of N-Terminus Through Anion-Exchange Column

Arginine deiminase modified with 20 kDa or 40 kDa PEG was purified through anion-exchange column chromatography (Bio-Rad Ltd.). The pH value of the mixed solution after reaction was adjusted to 7. Sample was loaded onto column pre-equilibrated in a equilibrium buffer containing 10 mM-Tris, pH 7.0. After loading the sample, the chromatography column was eluted with 3 column volume of equilibrium buffer, and then gradient elution was performed with buffer containing 10 mM Tris, 0-1 M NaCl, pH 7.0. The PEG which did not involve in reaction did not attach to the column but the peak thereof appeared during penetration and washing due to its minimal charge. The elution peaks appeared in the following order: multi-site modified arginine deiminase, mono-site specifically modified arginine deiminase, and unmodified arginine deiminase. Different fractions can be collected according to absorbance at 280 nm.

### Example 3

### The Significant Increase of the Half-Life in Blood of Arginine Deiminase Modified with PEG at a Single Site of N-Terminus

The half-life of arginine deiminase and PEG modified arginine in mice was measured respectively to evaluate the prolonged efficacy of the modification with PEG. 6 healthy Kunming mice (the average body weight is about 25 g) (Vitalriver Experimental Animal Center) were divided into 2 groups and injected with arginine deiminase and 20 kD PEG modified arginine deiminase via tail vein, in a dose of 15 mg/kg body weight. And then, blood samples were collected from tail vein at 2, 10, 30 minutes, 1, 2, 4, 8, 16, 24, 48, 72, 96, 120, 144 and 168 hours. Plasma was stored at minus 80° C. After blood was taken, the concentration of arginine deiminase and PEG modified arginine deiminase was measured through sandwich ELISA, respectively. In vivo pharmacokinetic result shows that in vivo half-life of arginine deiminase increases from an average of 4 hours to 72 hours after being modified with 20 kD PEG.

### Example 4

### Low Antigenicity of Arginine Deiminase Modified with PEG at a Single Site of N-Terminus in Experimental Animals

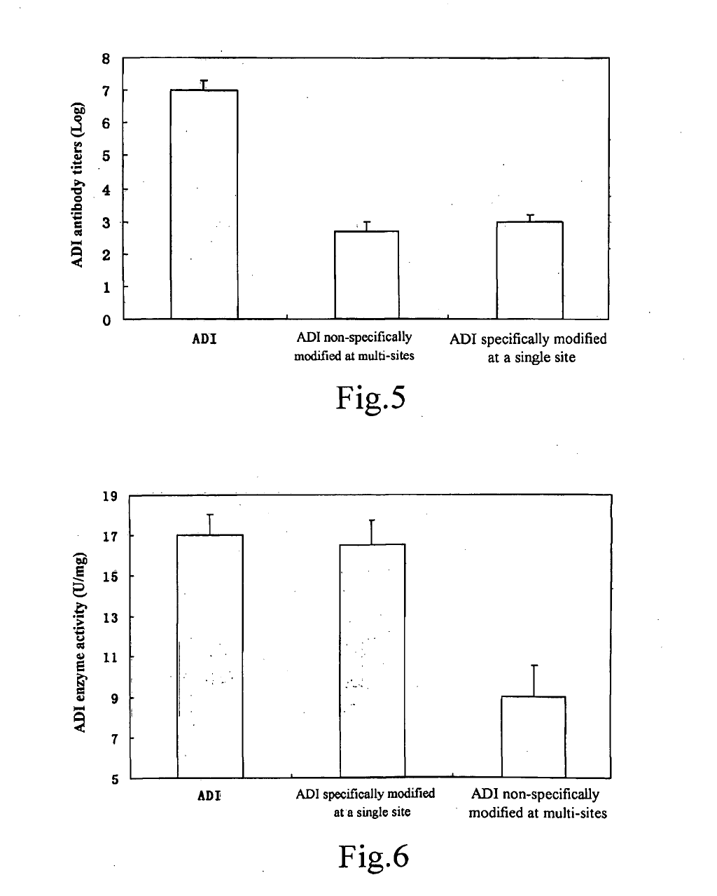

Immune responses induced by arginine deiminase, arginine deiminase specifically modified with PEG at a single site and arginine deiminase non-specifically modified at multi-sites were measured to examine the decrease of antigenicity after the modification with PEG at a single site (FIG. 5). 9 healthy Kunming mice (the average body weight is about 25 g) (Vitalriver Experimental Animal Center) were divided into 3 groups and injected with arginine deiminase, arginine deiminase specifically modified with 20 kD PEG at a single site of N-terminus and arginine deiminase non-specifically modified at multi-sites via tail vein, in a dose of 15 mg/kg body weight. Blood samples were collected from tail vein at 1, 7, 14, 21 days. Plasma was stored at minus 80° C. After blood was taken, antibody titers against arginine deiminase in the blood of mice of each group were measured respectively through sandwich ELISA. Immunological result shows that the antibody titer induced by arginine deiminase modified with 20 kD PEG at a single site of N-terminus is about 10,000 times lower than that induced by wild-type unmodified arginine deiminase, and is just 2-3 times higher than that induced by arginine deiminase non-specifically modified with PEG at multi-sites.

### Example 5

### Maintenance of the Integral Biochemical Activity of Arginine Deiminase Modified with PEG at a Single Site of N-Terminus

Arginine deiminase specifically modified with PEG at a single site was used to examine whether the enzyme activity decreased due to the modification (FIG. 6). Arginine deiminase was added to 100 ml phosphate buffered saline (PBS) to achieve a final concentration of 10 μg/ml, and then L-arginine was added as substrate of arginine deiminase to achieve a final concentration of 10 μM. The mixed reaction system was incubated for 10 minutes in a 37° C. bath, and the concentration change of L-arginine was measured by Blood Urea Nitrogen (BUN) kit (G-Cell Biotechnologies, Inc.). The result shows that the enzyme activities of unmodified arginine deiminase and arginine deiminase specifically modified at a single site are both 17 U/mg, which means that specific modification with 20 kDa PEG at a single site has no significant effect on the enzyme activity. We also used arginine deiminase non-specifically modified with PEG at multi-sites as a control. The result shows that the enzyme activity of arginine deiminase modified at multi-sites is only 50-60% of that of arginine deiminase modified with PEG at a single site of N-terminus under same molar concentration, which means that the enzyme activity of arginine deiminase non-specifically modified with PEG at multi-sites decreased significantly.

### Example 6

### The Activity of Inhibition of Tumor Cell Proliferation by Arginine Deiminase Modified with PEG at a Single Site of N-Terminus

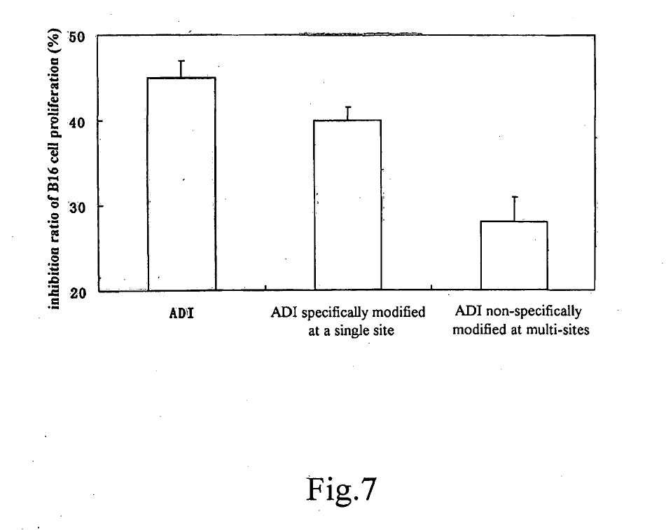

The inhibitory effect of 20 kDa PEG modified arginine deiminase on mice B 16/F 10 malignant melanoma cell proliferation was observed (FIG. 7). Mice malignant melanoma cells (B16/F10, ATCC # CRL-6475™, USA) were cultured to logarithmic growth phase in DMEM medium (Hyclone) containing 10% of serum, then starved in DMEM free of serum for 12 hours. Normal medium containing 10% fetal bovine serum and double antibiotics (10 μg/ml of streptomycin and ampicillin respectively, Sigma) was added. For treatment groups, arginine deiminase and modified arginine deiminase (including arginine deiminase specifically modified at a single site and arginine deiminase non-specifically modified at multi-sites) were added to achieve a final concentration of 10 μg/ml, while for control group, an equal volume of normal saline was added. After incubation at 37° C. for 24 hours, MTT was added to wells of the cell culture plate to achieve a final concentration of 0.25 mg/ml, and then the cells were incubated in 37° C. incubators (Thermo Electron Corporation) for 6 hours and dissolved by DMSO (Shanghai Sangon Biological Engineering Technology & Services Co., Ltd.) finally. The cell number was counted under microscope. Cells in three different fields on one plate were counted and then the inhibition rate was calculated. The result shows that inhibition rate of arginine deiminase on tumor proliferation is 40%, while inhibitory effect of arginine deiminase specifically modified at a single site on cell proliferation does not change. However, the enzyme activity of arginine deiminase modified at multi-sites is only about 70% of that of arginine deiminase modified at a single site, which means that the enzyme activity of PEG specifically modified arginine deiminase is maintained completely, and compared with deiminase modified at multi-sites, PEG specifically modified arginine deiminase can better inhibit tumor cell growth in vitro.

### Example 7

### Activity of Arginine Deiminase Modified with PEG at a Single Site of N-Terminus in the Treatment of Mice Tumor Models

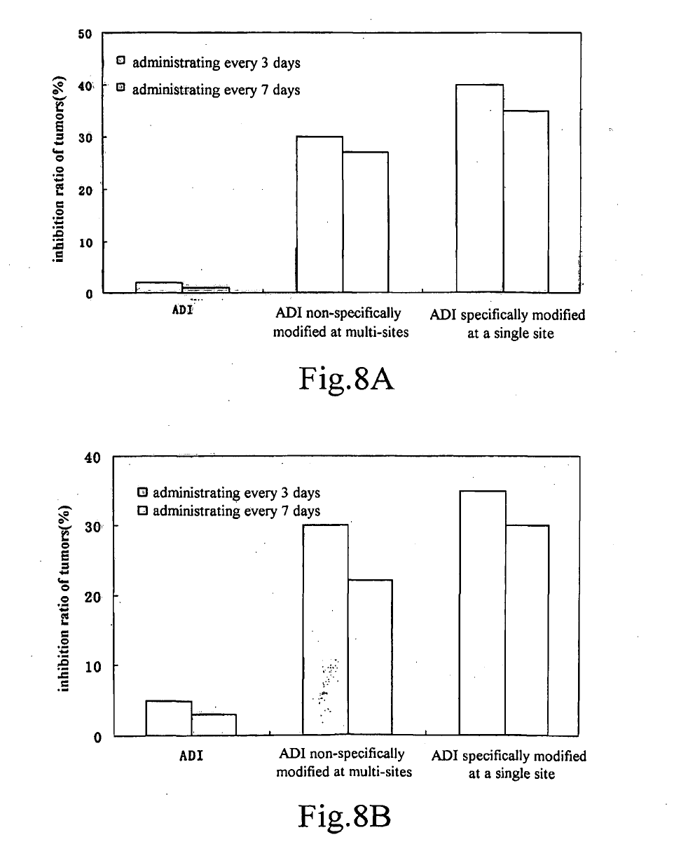

The in vivo inhibitory effect of 20 kDa PEG modified arginine deiminase on mice B16/F10 malignant melanoma was observed (FIG. 8A, B). Each C57 mice (Vitalriver Experimental Animal Center) with an average weight of about 20 g were injected with 2×10⁶ B16/F10 malignant melanoma cells via armpit. The mice were randomly grouped the next day, 8 mice per group. Designated were negative control group (normal saline), positive control group (arginine deiminase 5 mg/kg body weight (1.7 U/mouse), daily administration), and treatment groups which are employed to treatment of arginine deiminase specifically modified at a single site and arginine deiminase non-specifically modified at multi-sites, with an administration interval of once every 3 days and once every 7 days, respectively. Tumor-inoculated mice were administered after being divided into groups randomly via tail vein injection (FIG. 8A) and subcutaneous injection from back and neck (FIG. 8B) for 14 days. Then the mice were sacrificed, and tumor weights were measured on day 15. Tumor inhibition rate was calculated as follows to evaluate antitumor efficacy: tumor inhibition rate=(tumor weight of negative control group-tumor weight of treatment group)/tumor weight of negative control group x 100%. The result shows that the tumor inhibition rates of treatment group via tail vein injection per 3 days and per 7 days are 40% and 30% respectively; the tumor inhibition rates of treatment group via subcutaneous injection every 3 days and every 7 days are 35% and 30% respectively. Comparison of the tumor inhibition activities of arginine deiminase specifically modified at a single site and non-specifically modified at multi-sites shows that the tumor inhibition rate of arginine deiminase specifically modified at a single site is 10% higher than that of arginine deiminase non-specifically modified at multi-sites under the same experimental condition. The result shows that unmodified arginine deiminase has no significant activity of tumor inhibition; specifically modified arginine deiminase has better in vivo activity of tumor inhibition than arginine deiminase non-specifically modified at multi-sites; the antitumor efficacy of specifically modified arginine deiminase can be maintained under the extended administration interval.

### Example 8

### The Significant Effect of Arginine Deiminase Modified with PEG at a Single Site of N-Terminus on the Extension of Survival Time of Mice with Tumors

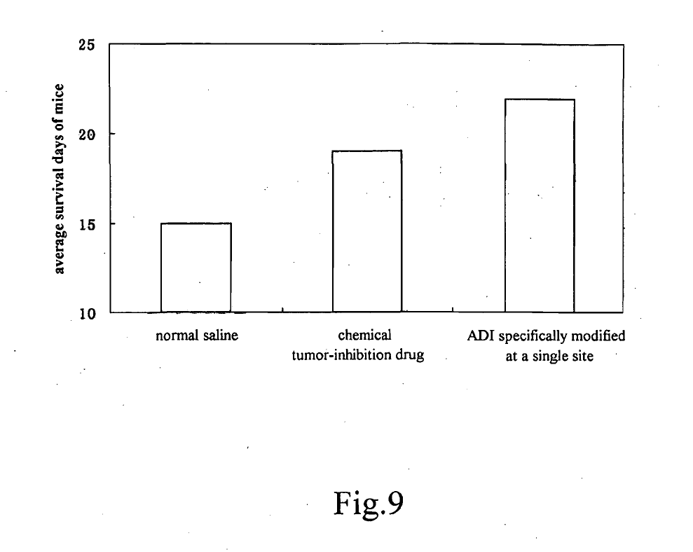

The treatment effect of arginine deiminase modified with 40 kD PEG at a single site on the survival time of C57 mice with B16/F10 malignant melanoma was observed (FIG. 9). Each C57 mice with an average weight of about 20 g were injected with 2×10⁶ B16/F10 malignant melanoma cells via armpit. The mice were randomly grouped the next day, 8 mice per group. Designated were the negative control (normal saline), the positive control (chemical tumor-inhibition drug, daily administration), the treatment group using arginine deiminase specifically modified at a single site (the interval of treatment was set once every 7 days), respectively. After being divided randomly into groups, tumor-inoculated mice were administered via subcutaneous injection from back and neck when the tumors reached an average diameter of about 2 cm. Treatment was performed over a period of 16 days, during which mice of each experimental group died one after another. The treatment effect was evaluated based on the average survival time of mice of each group. The result shows that the average survival time of mice of the negative control group, the positive control group and the treatment group is 15, 19 and 22 days, respectively.

1-47. (canceled)

48. A conjugate formed by coupling one polyethylene glycol molecule to one arginine deiminase molecule, wherein the polyethylene glycol molecule is coupled to the arginine deiminase at the N-terminal α-amino group of arginine deiminase or at the mercapto group of an additional cysteine residue attached to arginine deiminase, and the polyethylene glycol has an average molecular weight in the range from 5,000 to 100,000 Daltons, preferably 5,000 to 40,000 Daltons, and more preferably 20,000 to 40,000 Daltons.

49. The conjugate of claim 48, wherein said polyethylene glycol may be linear or branched.

50. The conjugate of claim 48, wherein the polyethylene glycol has the molecular weight of 20,000 or 40,000 Daltons.

51. The conjugate of claim 50, wherein polyethylene glycol is monomethoxy polyethylene glycol or monohydroxyl polyethylene glycol.

52. The conjugate of claim 51, wherein the monomethoxy polyethylene glycol derivative is monomethoxy polyethylene glycol butyrald (mPEG-ButyrALD), monomethoxy polyethylene glycol propionaldehyde, or monomethoxy polyethylene glycol succinimidyl propionic acid (mPEG-SPA).

53. The conjugate of claim 48, wherein the additional cysteine residue is a cysteine residue linked to the N-terminus, C-terminus, or the internal region of arginine deiminase molecule, or a cysteine residue on a peptide chain linked to the N-terminus, C-terminus, or the internal region of arginine deiminase molecule.

54. The conjugate of claim 48, wherein the arginine deiminase is derived from Mycoplasma hominis, Mycoplasma arthritidis, or Mycoplasma arginini, or the arginine deiminase is an arginine deiminase of Mycoplasma hominis, Mycoplasma arthritidis or Mycoplasma arginini prepared by cloning via genetic recombination technology.

55. The conjugate of claim 54, wherein the arginine deiminase has a sequence as shown in SEQ ID No.1 or SEQ ID No.2.

56. The conjugate of claim 48, wherein the arginine deiminase is a biologically active fragment, mutant, derivative, isomer of arginine deiminase or a combination thereof, preferably a biologically active fragment, mutant, derivative, isomer of arginine deiminase from Mycoplasma or a combination thereof, and more preferably a biologically active fragment, mutant, derivative, isomer of arginine deiminase from Mycoplasma hominis or a combination thereof.

57. The conjugate of claim 56, wherein the derivative of arginine deiminase has a sequence with an additive peptide of 1-15 amino acid residues in length at the N-terminus or C-terminus, preferably the derivative has a sequence as shown in SEQ ID No.3 or SEQ ID No.4.

58. A sustained-release formulation formed by a conjugate of claim 48 with a bio-compatible carrier, wherein said sustained-release formulation is in a form selected from the group consisting of microcapsule, hydrogel, microsphere, micro-osmotic pump or liposome.

59. A pharmaceutical composition comprising the conjugate of claim 48 or the sustained-release formulation in a form selected from the group consisting of microcapsule, hydrogel, microsphere, micro-osmotic pump or liposome and a pharmaceutically acceptable carrier.

60. The pharmaceutical composition of claim 59, wherein the pharmaceutically acceptable carrier is selected from the group consisting of aqueous pH buffer solutions including buffer solutions of phosphate, citrate and other organic acids; antioxidants including ascorbic acid; polypeptides with a low molecular weight (no more than 10 residues); proteins such as serum albumin, glutin or immunoglobulin; hydrophilic polymers such as polyvinylpyrrolidone; amino acids such as glycine, glutamine, asparagine, arginine or lysine; monosaccharide, disaccharide and other carbohydrates including glucose, mannose or dextrin; chelating agents such as EDTA; sugar alcohols such as mannitol or sorbitol; salt-forming counter ions such as sodium ion; nonionic surfactants such as TWEEN®, PEG and PLURONICS®.

61. A method for preventing or treating tumors, comprising administering the conjugate of claim 48 the sustained-release formulation in a form selected from the group consisting of microcapsule, hydrogel, microsphere, micro-osmotic pump or liposome, or the pharmaceutical composition comprising the conjugate of claim 48 or the sustained-release formulation in a form selected from the group consisting of microcapsule, hydrogel, microsphere, micro-osmotic pump or liposome and a pharmaceutically acceptable carrier to a subject.

62. The method of claim 61, the tumors are selected from the group consisting of lung cancer, hepatoma, gastric cancer, esophageal cancer, bone cancer, pancreatic cancer, lymphoma, colon cancer, breast cancer, prostate cancer, oral cancer, nasopharyngeal carcinoma, uterine cervix cancer, leukemia, malignant melanoma, sarcoma, renal cancer, biliary cancer or other tumors.

63. A method of preventing or treating diseases via reducing arginine content in a body or local tissue, comprising administering the conjugate of claim 48 the sustained-release formulation in a form selected from the group consisting of microcapsule, hydrogel, microsphere, micro-osmotic pump or liposome, or the pharmaceutical composition comprising the conjugate of claim 48 or the sustained-release formulation in a form selected from the group consisting of microcapsule, hydrogel, microsphere, micro-osmotic pump or liposome and a pharmaceutically acceptable carrier to a subject.

64. The method of claim 61, wherein the administration is selected from the group consisting of intravenous injection, intravenous drip administration, venous canal administration, arterial canal administration, intramuscular injection administration, oral administration, inhalation administration, subcutaneous administration, dermal administration, intraperitoneal administration, rectal administration, vaginal administration, nasal mucosa administration, oral mucosa administration, ocular administration.

65. A method for prolonging the in vivo half-life and anti-tumor efficacy of arginine deiminase, comprising forming a conjugate by coupling one polyethylene glycol molecule to one arginine deiminase molecule, wherein the coupling site is the N-terminal α-amino group of arginine deiminase or the mercapto group of an additional cysteine residue on arginine deiminase, or forming a sustained-release formulation of claim 58.

66. A conjugate formed by a modifying agent and an arginine deiminase, wherein said modifying agent is capable of enhancing the in vivo half-life of said arginine deiminase.
